(19) United States
(12) Patent Application Publication
(10) Pub. No.: US 2011/0039306 A1
(43) Pub. Date: Feb. 17, 2011

(54) Reagent Containing a Thermostable Endonuclease

(75) Inventors: Thomas C. Evans, JR., Topsfield, MA (US); Katherine Marks, Wenham, MA (US)

Correspondence Address: HARRIET M. STRIMPEL, D. Phil., New England Biolabs, Inc., 240 COUNTY ROAD, IPSWICH, MA, 01938-2723, US

(73) Assignee: NEW ENGLAND BIOLABS, INC., Ipswich, MA (US)

(21) Appl. No.: 12/988,844

(22) PCT Filed: Mar. 6, 2009
(86) PCT No.: PCT/US09/36363

§ 371 Date: Oct. 21, 2010

Related U.S. Application Data
(60) Provisional application No. 61/047,610, filed on Apr. 24, 2008.

(51) Int. Cl. C12P 19/34

(52) U.S. Cl. 435/91.2

(57) ABSTRACT

Compositions and methods are provided for preserving the thermostability of an endonuclease in the presence of DNA for uses that include DNA repair. The compositions and methods include the presence of zinc ions.

### BACKGROUND

E. coli endonuclease (Endo) IV is an apurinic/apyrimidinic (AP) endonuclease. While the literature refers to the enzyme as a metalloenzyme, the active enzyme tolerates a chelating agent. (Ljunquist & Lindahl, NAR 4:2871 (1977); Levin, et al, J. Biol. Chem. 266:22893 (1991)). Although E. coli Endo IV is active in the presence of a metal chelator, EDTA is known to destabilize E. coli Endo IV in the absence of its DNA substrate (Levin, et al., J. Biol. Chem. 263:8066 (1988)). AP endonucleases have been shown to be useful in DNA repair (US-2006-0088868, US-2006-0177867, WO 07/120,627). For this application at least, there are advantages to using a thermostable E. coli Endo IV provided that it is active during or following PCR.

### SUMMARY

An embodiment of the invention provides a reaction mixture that includes an endonuclease containing a sequence having at least 90% amino acid sequence homology with SEQ ID NO:1 and an effective amount of Zinc ions to preserve thermostability of the endonuclease in the presence of DNA wherein the amount of Zinc ions in the reaction mixture is at least 3 μM. An example of the endonuclease in the reaction mixture is an endonuclease that is derived from Thermus thermophilus (Tth).

In another embodiment of the invention, the reaction mixture contains zinc ions and DNA in a ratio of at least 1:20. The reaction mixture preferably has at least 60% activity for at least 30 minutes at 99° C., more specifically, at least 60% activity for at least 100 minutes at a temperature of at least 99° C.

In a further embodiment of the invention, a method is provided that includes the steps of: (a) adding DNA to a reaction mixture comprising a thermostable endonuclease (for example Tth) with zinc ions, the zinc ion concentration being at least 3 μM; (b) increasing the temperature of the reaction; and (c) preserving thermostability of the endonuclease in the presence of the DNA. The ratio of zinc ions to DNA may be more specifically identified as in the range of at least 1:20.

In a further embodiment of the method, the thermostable endonuclease has at least 90% sequence homology to SEQ ID NO:1.

In a further embodiment of the method, a reaction mixture as described herein is added to a preparation of damaged DNA so as to repair the DNA to the extent that permits amplification of the DNA by a polymerase chain reaction.

### BRIEF DESCRIPTION OF THE DRAWINGS

FIGS. 1A-1B show the thermostability of Thermus thermophilus (Tth) Endo IV in the absence of added Zn++. Acrylamide gel electrophoresis of a fluorescently labeled oligonucleotide was used to assess the presence of AP endonuclease activity.

FIG. 1A shows the results of serial dilution of the enzyme and heat treatment for 30 and 60 minutes.

Lane 1 shows the migration of the uncleaved oligonucleotide substrate used to assess the presence of AP endonuclease activity.

Lanes 2-8 show a serial dilution series of Tth Endo IV that was not heat treated. At the higher concentrations of Tth Endo IV, the oligonucleotide substrate is cleaved. At the lower concentrations of Tth Endo IV in the assay, there is observable uncleaved oligonucleotide substrate.

Lanes 9-14 show a serial dilution series of Tth Endo IV that had been heated to 99° C. for 30 minutes prior to assaying it.

Lanes 15-20 show a serial dilution series of Tth Endo IV that had been heated to 99° C. for 60 minutes prior to assaying it.

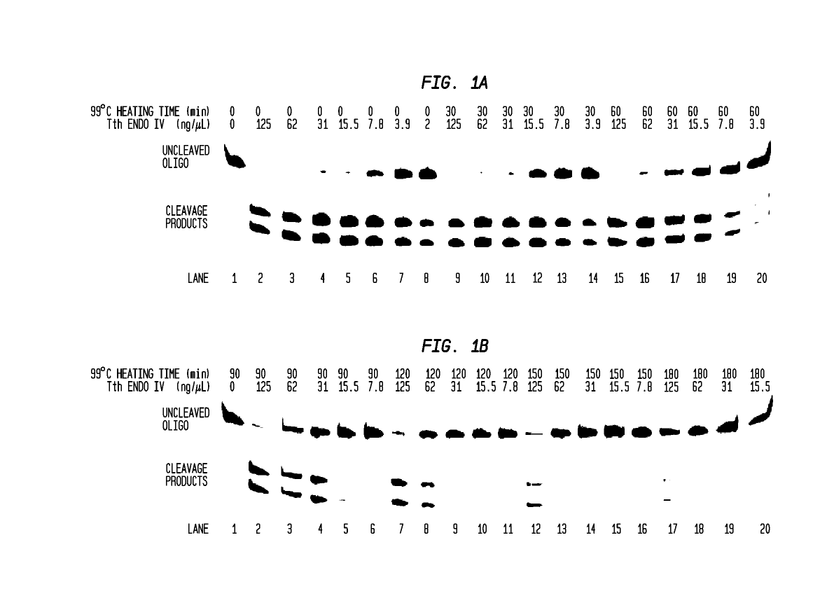

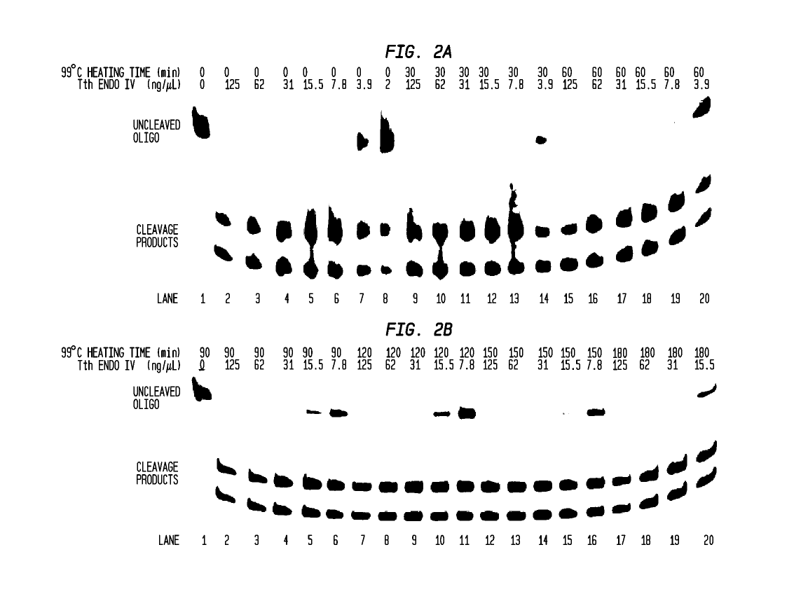

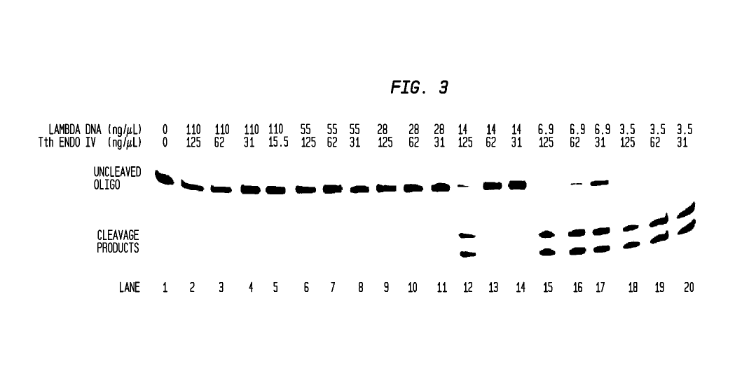

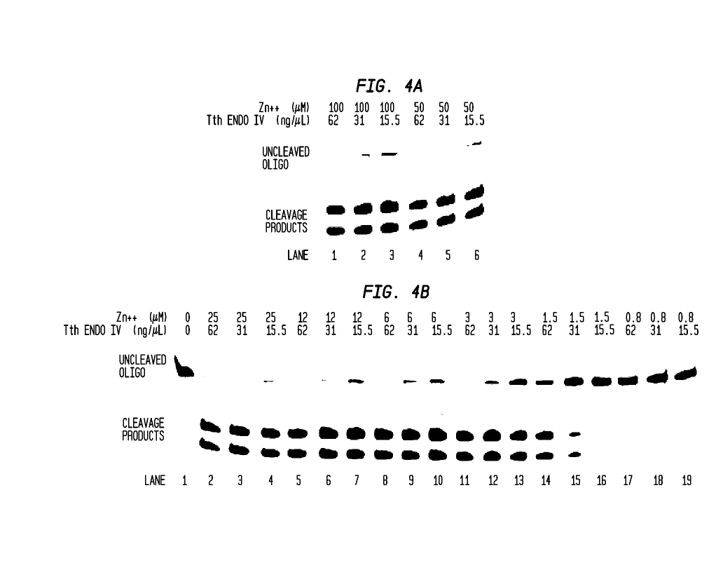

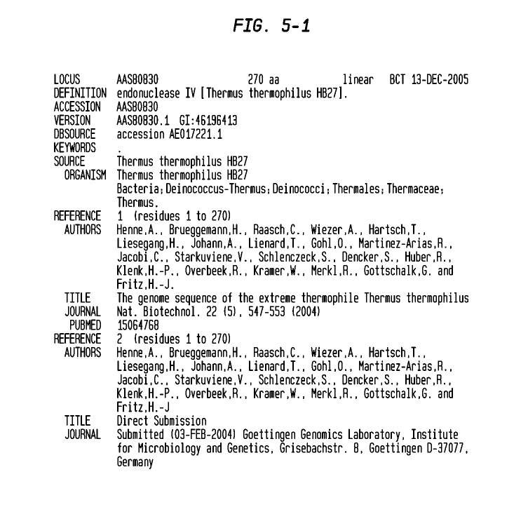

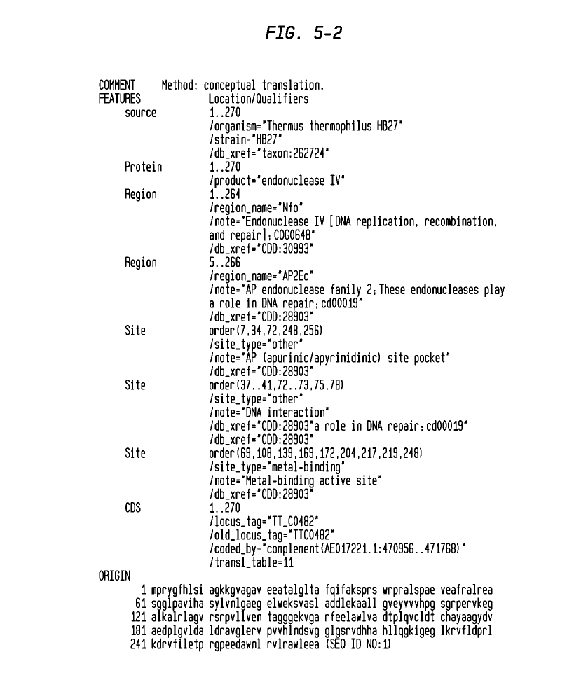

FIG. 1B shows serial dilutions of enzyme and heat treatment for 90, 120, 150 and 180 minutes.

Lane 1 shows the migration of the uncleaved oligonucleotide substrate used to assess the presence of AP endonuclease activity.

Lanes 2-6 show a serial dilution series of Tth Endo IV that was heated to 99° C. for 90 minutes prior to performing the AP endonuclease assay.

Lanes 7-11 show a serial dilution series of Tth Endo IV that had been heated to 99° C. for 120 minutes prior to assaying it.

Lanes 12-16 show a serial dilution series of Tth Endo IV that had been heated to 99° C. for 150 minutes prior to assaying it.

Lanes 17-20 show a serial dilution series of Tth Endo IV that had been heated to 99° C. for 180 minutes prior to assaying it.

FIGS. 2A-2B show the thermostability of Tth Endo IV in the presence of added Zn⁺⁺. Acrylamide gel electrophoresis of a fluorescently labeled oligonucleotide was used to assess the presence of AP endonuclease activity. The reactions were performed as in FIGS. 1A-1B, except that Zn⁺⁺ was added to the 99° C. heating reaction to a final concentration of 25 μM.

FIG. 2A shows the effect of 30 minutes and 60 minutes heat treatment on serial dilutions of enzyme in the presence of Zn⁺⁺.

Lane 1 shows the migration of the uncleaved oligonucleotide substrate used to assess the presence of AP endonuclease activity.

Lanes 2-8 show a serial dilution series of Tth Endo IV that was not heat treated. At the higher concentrations of Tth Endo IV, the oligonucleotide substrate was cleaved. At the lower concentrations of Tth Endo IV in the assay, there was observable uncleaved oligonucleotide substrate.

Lanes 9-14 show a serial dilution series of Tth Endo IV that had been heated to 99° C. for 30 minutes prior to assaying it.

Lanes 15-20 show a serial dilution series of Tth Endo IV that had been heated to 99° C. for 60 minutes prior to assaying it.

FIG. 2B shows the effect of 90, 120, 150 and 180 minutes heat treatment on serial dilutions of enzyme in the presence of Zn⁺⁺.

Lane 1 shows the migration of the uncleaved oligonucleotide substrate used to assess the presence of AP endonuclease activity.

Lanes 2-6 show a serial dilution series of Tth Endo IV that was heated to 99° C. for 90 minutes prior to performing the AP endonuclease assay.

Lanes 7-11 show a serial dilution series of Tth Endo IV that had been heated to 99° C. for 120 minutes prior to assaying it.

Lanes 12-16 show a serial dilution series of Tth Endo IV that had been heated to 99° C. for 150 minutes prior to assaying it.

Lanes 17-20 show a serial dilution series of Tth Endo IV that had been heated to 99° C. for 180 minutes prior to assaying it.

FIG. 3 shows that DNA counteracts the stabilizing influence of Zn⁺⁺ on Tth Endo IV. Acrylamide gel electrophoresis of a fluorescently labeled oligonucleotide was used to assess the presence of AP endonuclease activity. Tth Endo IV was subjected to thermocycling as described in the examples in the presence of 10 μM Zn⁺⁺ and varying concentrations of added DNA.

Lane 1 shows the uncleaved oligonucleotide substrate.

Lanes 2-5 show a titration of Tth Endo IV that had been subjected to thermocycling in the presence of 10 μM Zn⁺⁺ and 110 ng/μL Lambda DNA.

Lanes 6-8 show a titration of Tth Endo IV that had been subjected to thermocycling in the presence of 10 μM Zn⁺⁺ and 55 ng/μL Lambda DNA.

Lanes 9-11 show a titration of Tth Endo IV that had been subjected to thermocycling in the presence of 10 μM Zn⁺⁺ and 28 ng/μL Lambda DNA.

Lanes 12-14 show a titration of Tth Endo IV that had been subjected to thermocycling in the presence of 10 μM Zn⁺⁺ and 14 ng/μL Lambda DNA.

Lanes 15-17 show a titration of Tth Endo IV that had been subjected to thermocycling in the presence of 10 μM Zn⁺⁺ and 6.9 ng/μL Lambda DNA.

Lanes 18-20 show a titration of Tth Endo IV that had been subjected to thermocycling in the presence of 10 μM Zn⁺⁺ and 3.5 ng/μL Lambda DNA.

FIGS. 4A-4B show that additional Zn⁺⁺ stabilizes Tth Endo IV in the presence of DNA. Acrylamide gel electrophoresis of a fluorescently labeled oligonucleotide was used to assess the presence of AP endonuclease activity. Tth Endo IV was subjected to thermocycling as described in Example 3 in the presence of 2 ng/μL Lambda DNA and varying concentrations of Zn⁺⁺.

FIG. 4A shows the effect of varying amounts of zinc ions (100 μM, 50 μM) on the stability of the endonuclease.

Lanes 1-3 show Tth Endo IV titration in which 100 μM Zn⁺⁺ was present in the thermocycling reaction.

Lanes 4-6 show Tth Endo IV titration in which 50 μM Zn⁺⁺ was present in the thermocycling reaction.

FIG. 4B shows the effect of varying amounts of zinc ions (25 μM, 12 μM, 6 μM, 3 μM, 1.5 μM and 0.8 μM) on the stability of the endonuclease.

Lane 1 shows the uncleaved oligonucleotide substrate.

Lanes 2-4 show Tth Endo IV titration in which 25 μM Zn⁺⁺ was present in the thermocycling reaction.

Lanes 5-7 show Tth Endo IV titration in which 12 μM Zn⁺⁺ was present in the thermocycling reaction.

Lanes 8-10 show Tth Endo IV titration in which 6 μM Zn⁺⁺ was present in the thermocycling reaction.

Lanes 11-13 show Tth Endo IV titration in which 3 μM Zn⁺⁺ was present in the thermocycling reaction.

Lanes 14-16 show Tth Endo IV titration in which 1.5 μM Zn⁺⁺ was present in the thermocycling reaction.

Lanes 17-19 show Tth Endo IV titration in which 0.8 μM Zn⁺⁺ was present in the thermocycling reaction.

FIGS. 5-1 to 5-2 show the amino acid sequence data for Tth Endo IV (SEQ ID NO:1).

### DETAILED DESCRIPTION OF THE EMBODIMENTS

A reaction mixture is described herein that contains a thermostable endonuclease that retains its thermostability in the presence of DNA and zinc ions. The thermostable endonuclease in the reaction mixture has been defined as having at least 90% amino acid sequence homology with an amino acid sequence defined in SEQ ID NO:1 that corresponds to Tth Endo IV.

This is the first description of a thermophilic endonuclease in a reaction mixture where the reaction mixture renders the enzyme stable at temperatures above 90° C. more particularly at 99° C. temperatures. This stability profile renders the enzyme suitable for use under conditions of PCR. Although it might be concluded that a thermophilic endonuclease would withstand the temperature of incubation in a DNA preparation under conditions for PCR, FIG. 3, Lanes 2-5 show that Tth Endo IV in a thermopol buffer (New England Biolabs, Inc. (NEB), Ipswich, Mass.) is unstable during thermocycling in the presence of DNA. Surprisingly, the addition of Zn⁺⁺ results in stabilization of the enzyme in the presence of DNA. The benefit of adding zinc for stabilizing an enzyme in the presence of DNA substrate can be further enhanced by adding a non-ionic or ionic detergent such as Triton X-100. Other detergents known in the art may be used in place of Triton X-100.

The activity of an endonuclease is determined in units where one unit of enzyme is capable of cleaving 1 pmol of a 34mer oligonucleotide duplex containing a single AP site in a total reaction volume of 10 μl in 1 hour at 37° C. The activity of the Tth Endo IV was measured after 99° C. heat treatment or thermocycling in varying amounts of zinc ions and varying concentrations and at various temperatures in order to determine the concentrations required to create the novel reaction mixture. The results of activity assays are shown in FIGS. 1-4. These figures demonstrate that the preferred amount of zinc ions in the reaction mixture is at least 3 uM. The preferred ratio of zinc to DNA is at least 1:20.

All references cited herein, as well as U.S. provisional application Ser. No. 61/047,610 filed 24 Apr. 2008, are incorporated by reference.

### EXAMPLES

### Example 1

### Effect of Zn⁺⁺ on the Thermostability of Tth Endo IV

The heat stability of Tth Endo IV in the absence or presence of Zn⁺⁺ was evaluated by creating a master mix of 0.25 μg/μL of Tth Endo IV in a buffer composed of 30 Tris-HCl, 10 mM (NH₄)₂SO₄, 10 mM KCl, 25 mM NaCl, 4.5 mM MgSO₄, 0.1% Triton X-100, 12.5% glycerol, pH 8.3 at 25° C. 50 μL reactions of this mixture were incubated for the following time intervals: 0, 30, 60, 90, 120, 150 and 180 minutes at 99° C.

The activity of the enzyme after heat treatment was determined using a standard AP endonuclease assay. Tth Endo IV was identified by homology to E. coli Endo IV. Whether the enzyme behaved as expected by the homology was unknown because homology between proteins does not always result in the same behavior. The assay chosen to analyze Tth Endo IV activity was based on creating an abasic site in synthetic oligonucleotides of defined sequence. The ability of Tth Endo IV to cleave the abasic site-containing oligo was determined by running denaturing acrylamide gel electrophoresis.

Two synthetic oligonucleotides, U1, having the sequence C AAT ATA AGT AAA GTT ATA AAT AAA AAT GAA ATC (SEQ ID NO:2), and U2, having the sequence GAT TTC ATT TTT ATT UAT AAC TTT ACT TAT ATT GT (SEQ ID NO:3) were used in the AP endonuclease assay. Oligo U2 was labeled at the 5′ and 3′ ends with fluorescein to permit visualization on the acrylamide gel. The U in oligo U2 is a deoxyuracil (dU). Oligos U1 and U2 were annealed prior to use in the apurinic/apyrimidinic (AP) endonuclease assay.

The enzyme uracil DNA glycosylase (UDG) was included in the AP endonuclease assay. UDG specifically excises the uridine base from the dU leaving an AP site that can then be acted upon by an AP endonuclease activity. UDG does not act upon AP sites itself. Furthermore, UDG is present at a sufficient concentration that the dU is rapidly converted to an AP site.

A standard AP endonuclease assay contained the annealed U1 and U2 oligonucleotides at 0.25-0.5 pmol/μL, 0.05 units/μL UDG (NEB, Ipswich, Mass.), 1× thermopol buffer (20 mM Tris-HCl, 10 mM (NH₄)₂SO₄, 10 mM KCl, 2 mM MgSO₄, 0.1% Triton X-100, pH 8.8 at 25° C.) and the enzyme to be assayed. The enzyme concentration was serially diluted in a series of reactions. The reactions were incubated for 1 hour at 37° C. and quenched by adding formamide to 50% and SDS to 10% and 5× stop dye (12% ficoll, 0.01% bromphenol blue, 0.02% xylene, 7 M urea) to a concentration of 1×. The reactions were visualized by applying to a 20% TBE urea gel (30:1 acryl:bis). The oligonucleotide cleavage products were separated from intact oligonucleotides by the application of an electric potential gradient. The gels were scanned using a phosphoimager configured to detect fluorescein. Oligo U2 was fluorescein-labeled at both the 5′ and 3′ ends, which allowed for detection of the uncleaved oligo and the fragments that resulted from endonuclease cleavage (see FIGS. 1A and 1B). The oligo was cleaved at the dU position.

FIGS. 1A and 1B show that the Tth Endo IV has progressively less AP endonuclease activity as the incubation time at 99° C. increases. For example, note that almost no AP endonuclease activity was observed even at the highest enzyme concentration after a 180 minute incubation at 99° C. (FIG. 1B). In contrast to FIGS. 1A and 1B where almost no Tth Endo IV activity was visible after 180 minutes at 99° C., FIGS. 2A and 2B show that the Tth Endo IV displayed activity even at the lowest concentration assayed after 180 minute incubation at 99° C. This demonstrated that Zn⁺⁺ added to Tth Endo IV increased its thermostability.

### Example 2

### Thermostability of the Endonuclease was Reduced in the Presence of DNA

The heat stability of Tth Endo IV in the presence of 10 μM Zn⁺⁺ and varying concentrations of lambda DNA was evaluated. The reactions were composed of 0.25 μg/μL Tth Endo IV in a buffer of 30 mM Tris-HCl, 10 mM (NH₄)₂SO₄, 10 mM KCl, 25 mM NaCl, 4.5 mM MgSO₄, 10 μM ZnCl₂, 0.1% Triton X-100, 12.5% glycerol, and pH 8.3 at 25° C. The reactions further contained 110, 55, 28, 14, 6.9, or 3.5 ng/μL lambda DNA. The reactions were subjected to thermocycling using the following program: 1 cycle of 95° C. for 2 min and 40 cycles of 95° C. for 10 sec, 65° C. for 30 sec, and 72° C. for 1 min.

The amount of AP endonuclease activity remaining after thermocycling was determined using the AP endonuclease assay described in Example 1.

As can be seen in FIG. 3, the thermostability of the Tth Endo IV decreased when the DNA concentration was increased.

### Example 3

### An Effective Amount of Zn⁺⁺Ions Overcomes the Negative Impact of DNA on the Thermostability of Tth Endo IV

The heat stability of Tth Endo IV in the presence of 2 ng/μL lambda DNA and varying concentrations of Zn⁺⁺ was evaluated. The reactions were composed of 0.25 μg/μL Tth Endo IV and 2 ng/μL lambda DNA in a buffer of 30 mM Tris-HCl, 10 mM (NH₄)₂SO₄, 10 mM KCl, 25 mM NaCl, 4.5 mM MgSO₄, 0.1% Triton X-100, 12.5% glycerol, and pH 8.3 at 25° C. The reactions further contained 100, 50, 25, 12, 6, 3, 1.5, or 0.8 μM ZnCl₂. The reactions were subjected to thermocycling using the following program: 1 cycle of 95° C. for 2 min and 40 cycles of 95° C. for 10 sec, 65° C. for 30 sec, and 72° C. for 1 min.

The amount of AP endonuclease activity remaining after thermocycling was determined using the AP endonuclease assay described in Example 1.

As can be seen in FIG. 4, increasing the amount of Zn⁺⁺ in the reaction can overcome the negative effect of DNA on the thermostability of Tth Endo IV.

1. A reaction mixture, comprising: an endonuclease containing a sequence having at least 90% amino acid sequence homology with SEQ ID NO:1 and an effective amount of Zinc ions to preserve thermostability of the endonuclease in the presence of DNA wherein the amount of zinc ions in the reaction mixture is at least 3 μM.

2. A reaction mixture according to claim 1, wherein the ratio of zinc ions to DNA in the reaction mixture is at least 1:20.

3. A reaction mixture according to claim 2, wherein the endonuclease retains at least 50% activity for at least 10 minutes at 80° C.

4. A reaction mixture according to claim 2, wherein the endonuclease retains at least 60% activity for at least 30 minutes at 99° C.

5. A reaction mixture according to claim 2, wherein the endonuclease retains at least 60% activity for at least 100 minutes at a temperature of at least 99° C.

6. A reaction mixture according to claim 1, wherein the endonuclease is derived from Thermus thermophilus (Tth).

7. A method; comprising:
(a) adding DNA to a reaction mixture comprising a thermostable endonuclease with zinc ions, the zinc ion concentration being at least 3 μM;
(b) increasing the temperature of the reaction; and
(c) preserving thermostability of the endonuclease in the presence of the DNA.

8. A method according to claim 7, wherein the ratio of zinc ions to DNA is at least 1:20.

9. A method according to claim 7, wherein the endonuclease is derived from Tth.

10. A method according to claim 7, wherein the thermostable endonuclease has at least 90% sequence homology to SEQ ID NO:1.

11. A method, comprising:
(a) adding a reaction mixture according to claim 1 to a preparation of damaged DNA; and
(b) repairing the DNA to the extent that permits amplification of the DNA by a polymerase chain reaction.


SEQUENCE LISTING (3 sequences)

SEQ ID NO: 1 - protein, 270 aa, Thermus thermophilus
MPRYGFHLSIAGKKGVAGAVEEATALGLTAFQIFAKSPRSWRPRALSPAEVEAFRALREASGGLPAVIHASYLVNLGAEGELWEKSVASLADDLEKAALLGVEYVVVHPGSGRPERVKEGALKALRLAGVRSRPVLLVENTAGGGEKVGARFEELAWLVADTPLQVCLDTCHAYAAGYDVAEDPLGVLDALDRAVGLERVPVVHLNDSVGGLGSRVDHHAHLLQGKIGEGLKRVFLDPRLKDRVFILETPRGPEEDAWNLRVLRAWLEEA

SEQ ID NO: 2 - DNA, 34 bp, artificial
  oligo
caatataagtaaagttataaataaaaatgaaatc

SEQ ID NO: 3 - DNA, 35 bp, artificial
  oligo
gatttcatttttattuataactttacttatattgt